(19) United States
(12) Patent Application Publication
(10) Pub. No.: US 2016/0146534 A1
(43) Pub. Date: May 26, 2016

(54) METHOD FOR RECOVERING AN ETHYLENE STREAM FROM A CARBON MONOXIDE RICH FEED STREAM, AND ASSOCIATED INSTALLATION

(71) Applicant: TECHNIP FRANCE, Courbevoie (FR)

(72) Inventors: Bruno DESTOUR, Rueil Malmaison (FR); Yvon SIMON, Andresy (FR); Aurélia DADOU, Puteaux (FR)

(73) Assignee: TECHNIP FRANCE, Courbevoie (FR)

(21) Appl. No.: 14/900,433

(22) PCT Filed: Jun. 25, 2014
(86) PCT No.: PCT/EP2014/063424

§ 371 Date: Dec. 21, 2015

(30) Foreign Application Priority Data
Jun. 25, 2013 (FR) 13 56061

(51) Int. Cl. F25J 3/02; C07C 7/04; C07C 7/00

(52) U.S. Cl. CPC F25J 3/0238; C07C 7/005; C07C 7/04

(57) ABSTRACT

This method comprises the following steps:
treating a feed stream (12) to obtain a treated gas stream (60);
cooling the treated gas stream (60) in a heat exchanger (34) to form at least one column feed fraction;
feeding each column feed fraction into a distillation column (50);
heating at least one downstream flow (106) derived from the column head stream (104) in the heat exchanger (34).

The treatment step comprises the forming of an intermediate stream (20) containing at least 20 mole % ethylene and at least 20 mole % carbon monoxide, the method comprising a step to remove the carbon monoxide contained in the intermediate stream (20).

The present invention relates to a method for recovering an ethylene stream from a feed stream, comprising the following steps
treating a feed stream to obtain a treated gas stream;
cooling and at least partly condensing the treated gas stream in at least one heat exchanger to form at least one column feed fraction;
feeding the or each column feed fraction into a distillation column to recover an ethylene stream at the foot of the distillation column and an ethylene-depleted stream at the head of the column;
heating at least one downstream flow derived from the head stream in the heat exchanger.

Said method is particularly intended to treat a feed stream intended to produce ethylene, this feed stream originating from non-conventional ethylene sources. The feed stream comprises a high amount of carbon monoxide.

The production of light olefins such as ethylene is of considerable economic importance. These products are basic precursors for the polymer industry and are used to form plastic materials widely used in industry for the manufacture in particular of consumer goods, parts for transport vehicles or for the building sector.

At the current time, most industrially produced light olefins are sourced from the high temperature pyrolysis of hydrocarbons, in the presence of steam in particular.

The feed stream for pyrolysis essentially comprises ethane, propane, butane, naphtha and/or gas oil, alone or in a mixture.

The cracked gas output from pyrolysis comprises a mixture of water, ethylene, ethane, hydrogen, methane and other hydrocarbon compounds in variable proportions. In this type of production the obtained recovery rate and purity of the olefins produced are usually very high. Typically for ethylene the recovery rate is higher than 99° A), with purity higher than 99.5%.

However, other ethylene sources are currently under development. These sources produce a feed stream of very different composition to the stream usually output by steam cracking.

The feed stream may therefore comprise a high molar content of carbon monoxide e.g. higher than 10%, even higher than 20%.

In this case, the usual methods for recovering and treating the feed stream to obtain ethylene cannot be used or else they give low yields and recovery rates.

U.S. Pat. No. 6,303,841 describes a method for recovering and concentrating ethylene from a stream resulting from a conversion process of oxygenated compounds such as alcohols.

Said method has efficient ethylene recovery but requires complicated equipment and in particular a plurality of distillation columns.

It is one objective of the invention to provide a method for recovering ethylene from a feed stream originating from a non-conventional source, the method being simple to implement whilst guaranteeing very high recovery rate and ethylene purity.

For this purpose, the subject of the invention is a method of the aforementioned type, characterized in that the treatment step comprises the formation of an intermediate stream containing at least 20 mole % ethylene and at least 20 mole % carbon monoxide, the method comprising a step to remove the carbon monoxide contained in the intermediate stream.

The method of the invention may comprise one or more of the following characteristics taken alone or in any technically possible combination:
the pressure of the treated gas stream circulating in the or in each heat exchanger is lower than 40 kgf/cm², and in particular it is between 20 kgf/cm² and 40 kgf/cm²;
the pressure of the treated gas stream circulating in the or in each heat exchanger is strictly lower than 30 kgf/cm²;
the ethylene stream has a weight content of ethylene higher than 99.5%, the ethylene stream being obtained at the foot of the distillation column without passing through another distillation column;
the ethylene stream contains more than 99 weight % of the ethylene contained in the feed stream, the ethylene stream being obtained at the foot of the distillation column without passing through another distillation column;
at the treatment step, the intermediate stream is led into a carbon monoxide removing device without distillation to form a carbon monoxide-rich stream and a carbon monoxide-depleted stream, the pressure of the carbon monoxide-depleted stream advantageously being lower than 5 kgf/cm²;
the carbon monoxide removing device comprises a pressure swing adsorber producing the carbon monoxide-rich stream and the carbon monoxide-depleted stream;
the carbon monoxide-depleted stream is compressed to a re-compression pressure advantageously lower than 40 kgf/cm², in compression equipment to form the stream of treated gas;
at least one fraction of the treated gas stream is placed under heat exchange with the downstream flow in an upstream heat exchanger, to obtain a partly condensed upstream stream;
the upstream stream being led into an upstream separator, at least one liquid fraction output from the upstream separator is fed into the distillation column;
the temperature of the or of each column feed fraction is higher than −40° C.;
the intermediate stream is formed directly by the stream of treated gas, then led into a condensation and fractionating assembly comprising a plurality of heat exchangers operating at decreasing temperatures to produce a plurality of distillation column feed fractions fed into the distillation column, the head stream derived from the distillation column containing more than 10 weight % of the carbon monoxide contained in the intermediate stream;
the method comprises the following steps:
leading the treated gas stream into at least one first upstream heat exchanger to obtain a cooled, party condensed upstream stream at a temperature advantageously lower than −50° C., and separating the upstream stream into an upstream column feed liquid fraction and a downstream gas fraction;
leading the upstream gas fraction into at least one intermediate heat exchanger to obtain at least one cooled and partly condensed intermediate stream at a temperature advantageously lower than −90° C., and separating the or each cooled intermediate stream into an intermediate column feed liquid fraction and an intermediate gas fraction;
leading at least one intermediate gas fraction into a downstream heat exchanger to obtain a cooled, partly condensed downstream stream at a temperature advantageously lower than −110° C., and separating the downstream stream into a downstream column feed liquid fraction and a downstream gas fraction, the upstream, downstream and intermediate liquid fractions being fed into the distillation column;
the passing of at least part of the downstream gas fraction into the downstream heat exchanger and/or into the intermediate heat exchanger and/or into the upstream heat exchanger for heating therein, the method comprising a pressure-reducing step of the heated downstream gas fraction in a dynamic expansion turbine or static pressure-reducing valve before being sent back to the downstream heat exchanger and/or intermediate heat exchanger and/or upstream heat exchanger;
a step to circulate the downstream flow derived from the head stream in the upstream heat exchanger and/or in the intermediate heat exchanger and/or in the downstream heat exchanger;
at least one recirculation flow derived from the downstream flow, after heating the downstream flow in the heat exchanger, is sent back after compression to the feed stream, upstream of the treatment step;
at least one recirculation flow derived from the downstream flow, after heating of the downstream flow in the heat exchanger, is sent back to the treated gas stream downstream of the treatment step to form the intermediate stream.

A further subject of the invention is an installation for recovery of an ethylene stream from a feed stream, the installation comprising:
an assembly for treatment of a feed stream to obtain a treated gas stream;
an assembly for cooling and at least partly condensing the treated gas stream, comprising at least one heat exchanger to form at least one column feed fraction;
a distillation assembly comprising a distillation column and an arrangement to feed the or each column feed fraction into the distillation column so as to recover an ethylene stream at the foot of the distillation column and a head stream depleted of ethylene;
an assembly to heat at least one downstream flow derived from the head stream in the heat exchanger;

characterized in that the treatment assembly comprises an arrangement to form an intermediate stream containing at least 20 mole % ethylene and at least 20 mole carbon monoxide, the installation comprising an assembly to remove the carbon monoxide contained in the intermediate stream; the distillation assembly being configured to remove at least part of the carbon monoxide contained in the intermediate stream.

The installation of the invention may comprise one of more of the following characteristics taken alone or in any technically possible combination:
the removing assembly comprises a device to remove carbon monoxide without distillation, to form a carbon monoxide-rich stream and a carbon monoxide-depleted stream, the removing device being capable of producing the carbon monoxide-depleted stream at a pressure advantageously lower than 5 kgf/cm²;
the removing assembly is formed by the distillation assembly.

The invention will be better understood on reading the following description given solely as an example and with reference to the appended drawings in which:

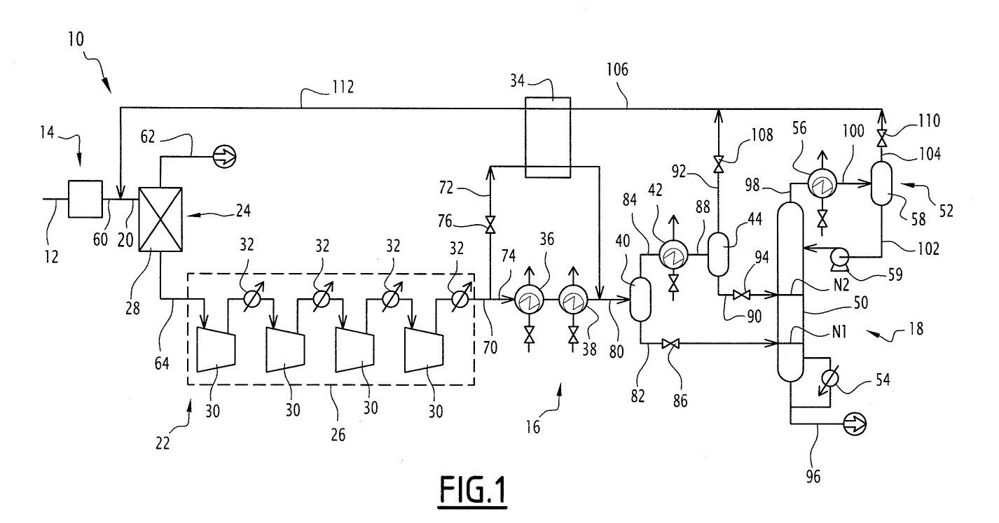

FIG. 1 is a functional synoptic diagram of a first installation intended for implementation of a first ethylene recovery method according to the invention;

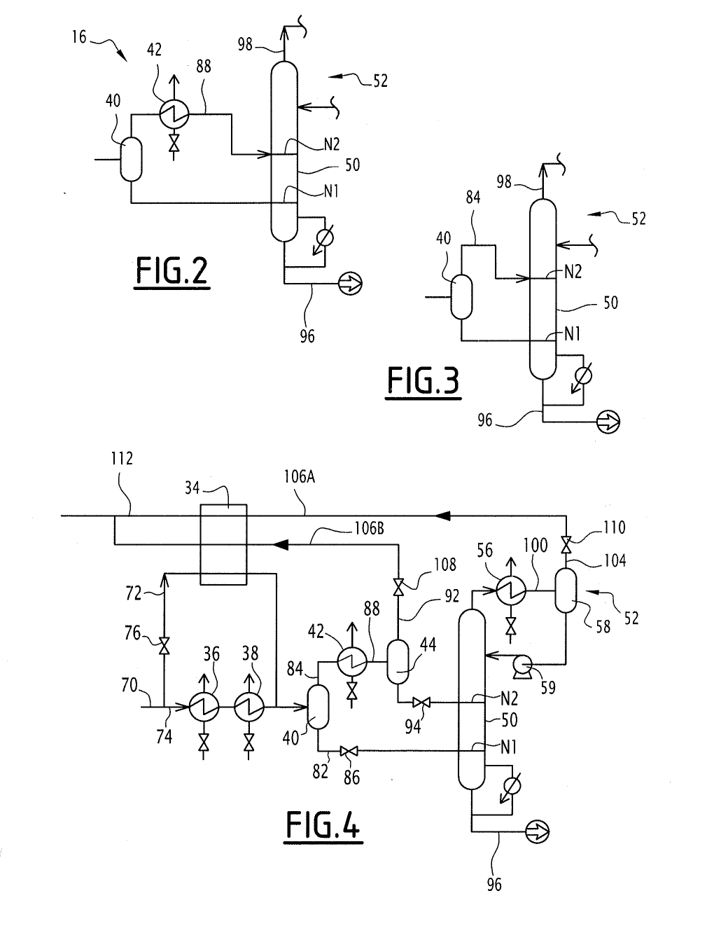

FIG. 2 gives a view of one detail of a first variant of the first installation according to the invention;

FIG. 3 gives a view of a detail of a second variant of the first installation of the invention;

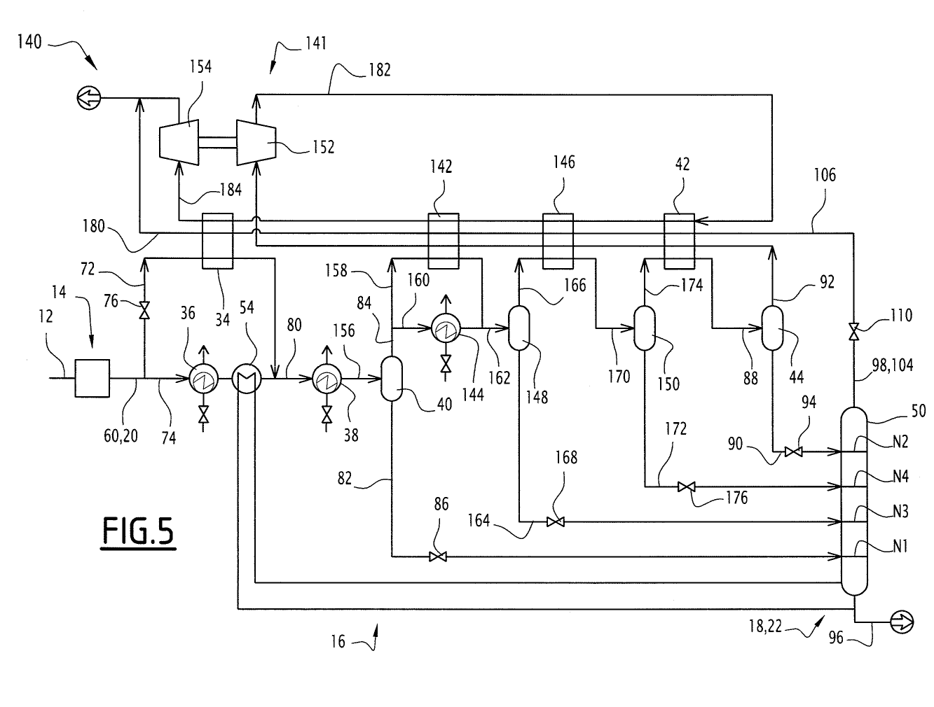

FIG. 4 gives a view of a detail of a third variant of the first installation according to the invention;

FIG. 5 is a functional synoptic diagram of a second installation intended for implementation of a second ethylene recovery method according to the invention;

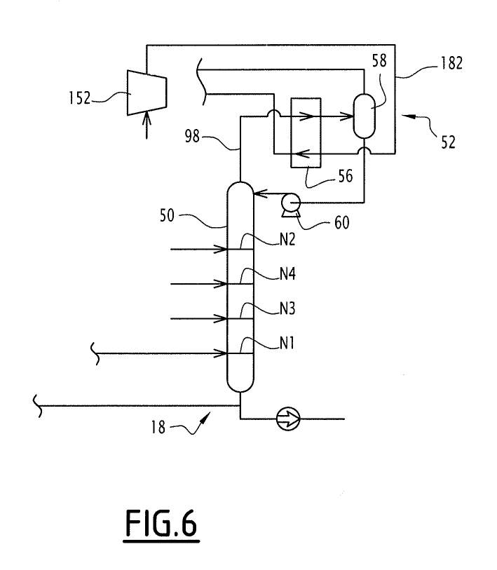

FIG. 6 is a view of a detail of a first variant of the second installation according to the invention.

In the remainder hereof, one same reference designates a stream circulating in a duct and the duct carrying this stream. Unless otherwise indicated, percentages are mole percentages, temperatures and pressures are respectively in relative degrees Celsius and kilogram-force per square centimetre (kgf/cm²).

A first installation 10 to recover ethylene from a gas feed stream 12 is illustrated in FIG. 1.

The feed stream 12 is obtained from non-conventional ethylene sources and not from a high temperature hydrocarbon cracking process in the presence of steam.

The feed stream 12 has a molar content of ethylene higher than 20% and between 20% and 80%, advantageously between 40% and 60%. It has a molar content of carbon monoxide higher than 20% and between 20% and 80%, typically between 40% and 60%.

The molar content of methane in the feed stream 12 is lower than 20%. The molar content of hydrogen in the feed stream 12 is typically less than 10%.

The feed stream 12 contains impurities such as acid gases, in particular carbon dioxide (CO₂) and potentially hydrogen sulfide (H₂S) or other impurities such as oxygenated compounds and water.

The installation 10 comprises an assembly 14 to treat the feed gas 12 intended to form a purified feed stream, a removing assembly 22 to pre-fractionate the purified gas, an assembly 16 to cool and at least partly condense a treated gas obtained from the feed stream, and a distillation assembly 18.

The treatment assembly 14 is able to remove the impurities contained in the feed stream 12 for at least partial forming of an intermediate stream 20 rich in carbon monoxide.

For this purpose, the treatment assembly 14 is capable of generating a purified feed stream 60 no longer containing impurities such as acid gases, in particular carbon dioxide (CO₂) and potentially hydrogen sulfide (H₂S) or other impurities such as oxygenated compounds other than carbon monoxide (CO) and water.

For example the treatment assembly 14 comprises a caustic soda scrub tower able to remove acid gases, or an amine scrub tower.

The treatment assembly 14 also comprises drying molecular sieves for example able to remove water. The treatment assembly 14 may also contain catalyst or trap beds to remove the impurities in the gas, heavy metals in particular.

As will be seen below, the intermediate stream 20 rich in carbon monoxide is formed in the treatment assembly 14 from the purified feed stream 60.

The removing assembly 22 is particularly intended to remove the carbon monoxide contained in the intermediate stream 20.

In this example, the removing assembly 22 is positioned between the treatment assembly 14 and the condensation assembly 16.

In the example illustrated FIG. 1, the removing assembly 22 comprises a device 24 for removing at low pressure of the carbon monoxide contained in the intermediate stream 20, and a compression device 26 intended to form the treated gas stream.

Preferably in this variant, the removing device 24 comprises equipment 28 to adsorb carbon monoxide by pressure modulation known as a pressure swing adsorber—PSA.

Said equipment 28 operates at low purge pressure e.g. lower than 10 kgf/cm² and in particular between 0.1 kgf/cm² and 5 kgf/cm². From the intermediate stream 20 it is capable of producing a stream 62 depleted of ethylene containing less than 1 mole % of the ethylene contained in the intermediate stream 20, and more than 60 mole % of the carbon monoxide contained in the intermediate stream 20, advantageously more than 80%.

The pressure swing adsorber 28 comprises at least two enclosures operating in turn under ethylene adsorption conditions on a substrate at relatively high pressure, and ethylene desorption at relatively low pressure to release the ethylene adsorbed on the substrate.

For example the adsorption substrate comprises one or more molecular sieve beds, in particular beds of zeolites and/or aluminosilicates and/or microporous carbon.

The compression device 26 comprises a compressor itself formed of a plurality of compression stages 30 mounted in series, and a plurality of refrigerants 32 mounted at the output of each compression stage 30 to cool the compressed gas output from the compressor 30.

The cooling and condensation assembly 16 comprises at least one upstream heat exchanger 34, 36, 38, to form a partly condensed upstream gas stream, and an upstream separator 40 to separate the upstream gas stream.

The cooling and condensing assembly 16 in this example comprises at least one downstream heat exchanger 42 to form a partly condensed downstream gas stream, and a downstream separator 44 to separate the downstream gas stream.

In this example, the refrigeration and condensing assembly 16 comprises a first upstream heat exchanger 34, a second upstream heat exchanger 36 and a third upstream heat exchanger 38.

The second upstream heat exchanger 36 and the third upstream heat exchanger 38 are mounted in series. They are connected to a refrigeration loop using a refrigerant in a refrigeration cycle (not illustrated). The refrigerant is advantageously a hydrocarbon such as propylene.

The distillation assembly 18 comprises a distillation column 50, a head condenser 52 and a bottom reboiler 54.

In this example, the column 50 is a distillation column with reboiled stripper column.

The distillation column 50 is able to operate at a pressure of between 10 kgf/cm² and 40 kgf/cm², in particular between 25 kgf/cm² and 40 kgf/cm², preferably between 30 kgf/cm² and 40 kgf/cm².

It comprises plates or a lining. For example it contains more than 6 theoretical plates and in particular between 10 theoretical plates and 20 theoretical plates.

The head condenser 52 comprises a head heat exchanger 56, a head separator 58, and a reflux pump 59.

A first method to recover an ethylene stream from a feed stream 12 carried out in the first installation 10, is now described.

Initially, the feed stream 12 held at a pressure advantageously between 10 kgf/cm² and 40 kgf/cm² is fed into the treatment assembly 14. It advantageously has a temperature of between 10° C. and 50° C. The feed stream 12 is rich in ethylene and carbon monoxide. It preferably has the above-described composition.

A purified feed stream 60 free of impurities is extracted from the treatment assembly 14.

The purified feed stream 60 has a molar content of acid gases such as carbon dioxide (CO₂) and hydrogen sulfide (H₂S)] of less than 1 ppm (0.0001 mole %), and a water content less than 1 ppm (0.0001 mole %).

The purified feed stream 60 at least partly forms the intermediate stream 20.

The molar content of ethylene in the intermediate stream 20 is higher than 20%, and in particular it is between 20% and 80%, preferably between 40% and 60%. The molar content of carbon monoxide in the intermediate stream 20 is higher than 20% and in particular it is between 20% and 80%, preferably between 40% and 60%.

The intermediate stream 20 is then fed into the removing device 24. A carbon monoxide-rich stream 62 is then continuously extracted at high pressure from the removing device 24. A stream 64 depleted of carbon monoxide is simultaneously and continuously extracted at low pressure from the removing device 24.

For this purpose, the intermediate stream 20 successively passes in one of the enclosures of the adsorption equipment 28 to allow adsorption of the ethylene contained in the stream 20 on the substrate contained in the enclosure, and the formation of a stream 62 rich in carbon monoxide. Simultaneously the ethylene loaded on the substrate contained in the other enclosure desorbs and forms the carbon-monoxide depleted stream 64.

The carbon monoxide-rich stream 62 has a pressure higher than 10 kgf/cm² and in particular of between 20 kgf/cm² and 40 kgf/cm². It comprises more than 60 mole % of the carbon monoxide contained in the intermediate stream 20, advantageously more than 80 mole %.

The carbon-monoxide depleted stream 64 has a pressure lower than 10 kgf/cm² and in particular of between 0.1 kgf/cm² and 5 kgf/cm². It contains more than 99 mole % of the ethylene contained in the intermediate stream 20, and advantageously less than 20 mole % of carbon monoxide.

The carbon-monoxide depleted stream 64 is then fed into the compression device 26. It is compressed in the successive compression stages 30 up to the operating pressure of the column 50, being cooled at each compression stage in a refrigerant 32.

The treated gas stream 70 recovered at the output of equipment 26 advantageously has a pressure of between 10 kgf/cm² and 40 kgf/cm², and in particular between 25 kgf/cm² and 40 kgf/cm². The pressure of the flow 70 is controlled by a valve 108 located downstream of the downstream separator 44.

The temperature of the treated gas stream 70 is higher than 10° C. for example, in particular it is between 20° C. and 50° C.

The treated gas stream 70 is then led into the condensation assembly 16 to be at least partly condensed therein in exchangers 36, 38, 34.

In this example, the treated gas stream 70 is separated into a first fraction 72 and a second fraction 74.

The first fraction 72 is led into the first heat exchanger 34 to be cooled therein down to a temperature lower than −10° C., and in particular of between −15° C. and −25° C.

The second fraction 74 is successively led into the second heat exchanger 36 and third heat exchanger 38 to be cooled therein via heat exchange with the refrigerant circulating in the external refrigeration cycle e.g. with propylene with refrigerant vaporization.

The second fraction 74 is cooled down to a temperature lower than −10° C. and in particular of between −15° C. and −25° C.

The ratio of the molar flow rate of the first fraction 72 to the second fraction 74 is controlled by a valve 76 as a function of the negative kilocalories available in the first heat exchanger 34.

The cooled first fraction 72 and cooled second fraction 74 are then grouped together to form an at least partly condensed upstream stream 80.

The molar content of liquid in the upstream stream 80 is higher than 40% for example and in particular it is between 50% and 70%. The upstream stream 80 is then led into the upstream separator 40 to be separated therein into an upstream liquid fraction 82 and an upstream gas fraction 84.

The upstream liquid fraction 82 forms a first feed fraction of the column 50 fed into the column 50 at a first level N1 through a flow control valve 86.

The upstream gas fraction 84 is then led into the first downstream heat exchanger 42 to be cooled and partly condensed therein via heat exchange with a refrigerant circulating in an external refrigeration cycle and to form a downstream stream 88.

The temperature of the downstream stream 88 is lower than −20° C. and in particular it is between −25° C. and −40° C.

The molar content of liquid in the downstream stream 88 is higher than 30% for example and in particular it is between 40% and 60%.

The downstream stream 88 is then led into the downstream separator 44 to be separated therein into a downstream liquid fraction 90 and a downstream gas fraction 92.

The downstream liquid fraction 90 forms a second feed fraction for the column 50 which is fed into the column 50 at a second level N2 located above the first level N1 through a flow control valve 94.

The pressure of the column 50 is lower than 40 kgf/cm², and in particular it is between 25 kgf/cm² an 40 kgf/cm². The pressure of the column 50 is controlled by a valve 110 located downstream of the head separator 58.

The column 50 is heated via a bottom reboiler 54 in which there circulates a stream which may typically be a condensing refrigerant circulating in an external refrigeration cycle intended to supply one of the exchangers 36, 38, 42.

An ethylene-rich foot stream 96 is recovered at the foot of the column 50. The foot stream 96 has a molar ethylene content higher than 99.5%, and carbon monoxide molar content of less than 0.0001 mole % (1 molar ppm). The foot stream 96 contains more than 99 mole % of the ethylene contained in the feed stream 12.

Therefore the foot stream 96 can be used directly in a polymer production unit without having to be re-distilled.

A head stream 98 depleted of ethylene is extracted at the head of the column 50.

The head stream 98 is partly condensed in the head exchanger 56 via heat exchange with a refrigerant (typically vaporizing propylene) circulating in a conventional refrigeration loop in a closed refrigeration cycle.

The partly condensed head stream 100 is then led into the head separator 58 to be separated therein into a head liquid fraction 102 and a head gas fraction 104.

The head liquid fraction 102 is pumped via a reflux pump 59 into the column 50.

The head gas fraction 104 is then mixed with the downstream gas fraction 92 output from the downstream separator 44 to form a head downstream flow 106 derived from the head stream 98.

The temperature of the downstream flow 106 upstream of the downstream exchanger 34 is between −25° C. and −40° C. for example. The pressure of the downstream flow 106 is equal to the pressure of the pressure of the flow 60 plus the head loss generated in the heat exchanger 34. For example this pressure is between 20 kgf/cm² and 40 kgf/cm².

The downstream flow 106 is then led into the first heat exchanger 34 to be heated via heat exchange with the first fraction 72 of the treated gas stream 70.

The heated downstream flow 112 output from the exchanger 34 has a temperature higher than 0° C. It is then at least partly fed back into the treated feed stream 60 to form the intermediate stream 20.

With the method of the invention it is therefore possible in simple and particularly economical manner to treat a feed stream 12 sourced from a non-conventional ethylene source, and to extract therefrom the entirety of the ethylene and to produce a stream 96 meeting specifications for polymer production.

This result is obtained with a single distillation step performed in a single column 50 and using an easily-operated carbon monoxide removing assembly 22.

The recovery of ethylene from the feed stream 12 is practically total e.g. higher than 99%, and the purity of the ethylene obtained is higher than 99.5° A.

In a first variant of the first installation 10 partly illustrated FIG. 2, the condensation assembly 16 does not have a downstream separator 44.

The downstream stream 88 cooled and at least partly condensed in the downstream exchanger 42 is fed directly into the column 50 at level N2, above feed level N1 for the first column feed fraction.

The recovery method is implemented in this variant of installation 10 is similar to the method implemented in the first installation 10.

In a second variant of the first installation 10, partly illustrated FIG. 3, the condensation assembly 16 is also devoid of downstream separator 44 and is devoid of a downstream heat exchanger 42.

The upstream gas fraction 84 output from the downstream separator 40 is led directly into the column 50 to form the second column feed fraction.

The recovery method implemented in this variant of installation 10 is also similar to the method implemented in the first installation 10.

In a third variant of the first installation 10, partly illustrated FIG. 4, the head gas fraction 104 derived from valve 110, and the downstream gas fraction 92 derived from valve 108 respectively form a first downstream flow 106A and a second downstream flow 106B which are separately led into the first heat exchanger 34 to be heated therein before being mixed with one another downstream of the first heat exchanger 34.

The recovery method implemented in this variant of installation 10 is also similar to the one implemented in the first installation 10.

In one variant (not illustrated) the head heat exchanger 56 (not illustrated) is a vertical heat exchanger arranged in the column 50.

A second installation 140 according to the invention is illustrated FIG. 5.

Like the first installation 10, the second installation 140 comprises an assembly 14 to treat the feed gas 12 intended to form a treated gas, an assembly 16 to cool and at least partly condense the treated gas and a distillation assembly 18.

According to the invention, the treatment assembly 14 is able to generate a carbon monoxide-rich intermediate stream 20.

However, unlike installation 10, the assembly 22 to remove the carbon monoxide contained in the intermediate stream 20 is formed directly by the distillation assembly 18.

The installation 140 further comprises an additional refrigeration assembly 141 for the treated feed stream 20.

The treatment assembly 14 is similar to that of the first installation 10. It will not be further detailed

As for the first installation 10, the refrigeration and condensing assembly 16 comprises an upstream stage comprising at least one upstream heat exchanger 34, 36, 38 to form a partly condensed upstream gas stream, and an upstream separator 40 to separate the upstream gas stream.

The condensation assembly 16 also comprises a downstream stage comprising at least one downstream heat exchanger 42, to form a partly condensed downstream gas stream, and a downstream separator 44 to separate the downstream gas stream.

Unlike in the first installation 10, the condensation assembly 16 of the second installation further comprises an intermediate stage comprising intermediate heat exchangers 142, 144, 146, and intermediate separators 148, 150 respectively arranged downstream of the intermediate heat exchangers 142, 144 and of intermediate heat exchanger 146.

In this example, the first intermediate heat exchanger 142 is mounted in parallel with the second intermediate heat exchanger 144.

The first intermediate heat exchanger 142 is able to be cooled by heating a downstream flow 106 obtained from a head stream 98 formed in the distillation assembly 18.

The first intermediate separator 148 is positioned between exchangers 142, 144 and exchanger 146.

The second intermediate heat exchanger 144 is able to be cooled via vaporization of a refrigerant circulating in a closed refrigerating cycle (not illustrated). The refrigerant may be ethylene for example.

The third intermediate heat exchanger 146 is able to be cooled by heating a downstream flow 106 obtained from a head stream 98 formed in the distillation assembly 18.

In the example illustrated FIG. 5, the distillation assembly 18 is devoid of a head condenser 52. The distillation column 50 is an absorption column with reboiling.

The reboiler 54 is positioned downstream of the first upstream heat exchanger 36, and upstream of the second upstream heat exchanger 38, so as to be placed in heat exchange contact with the second fraction 74.

The additional refrigeration assembly 141 comprises a dynamic expansion turbine 152 coupled to a compressor 154. The dynamic expansion turbine 152 is able to receive at least part of the downstream gas flow for expansion and circulation thereof through the heat exchangers 34, 142, 146, 42 and to provide negative calories to cool the treated gas stream 20.

A second method of the invention implemented in the second installation 140 will now be described.

Initially the feed stream 12, held at a pressure advantageously between 10 kgf/cm² and 40 kgf/cm², in particular between 20 kgf/cm² and 30 kgf/cm², is fed into the treatment assembly 14. It advantageously has a temperature between 10° C. and 50° C.

The feed stream 12 is rich in ethylene and carbon monoxide. It preferably has the above-described composition.

A treated feed stream 60, free of impurities, is extracted from the treatment assembly 14. The feed stream 60 has a molar content of acid gases such as carbon dioxide (CO₂) and hydrogen sulfide (H₂S)] of less than 1 molar ppm (0.0001 mole %), and a molar content of water of less than 1 molar ppm (0.0001 mole %).

In this example, the treated feed stream 60 forms the carbon monoxide-rich intermediate stream 20.

The molar content of ethylene in the intermediate stream 20 is higher than 20% and in particular it is between 20% and 80%, preferably between 40% and 60%. The molar content of carbon monoxide in the intermediate stream 20 is higher than 20% and in particular it is between 20% and 80%, preferably between 40% and 60%.

The intermediate stream 20 advantageously has a pressure between 10 kgf/cm² and 40 kgf/cm², in particular between 20 kgf/cm² and 35 kgf/cm², and more particularly lower than 30 kgf/cm².

The temperature of the intermediate stream 20 is higher than 10° C. for example and in particular it is between 20° C. and 50° C.

The intermediate stream 20 is then led into the condensation assembly 16 to be at least partly condensed therein in exchangers 36, 38, 34, 54.

In this example, the intermediate stream 20 is separated into a first fraction 72 and a second fraction 74.

The first fraction 72 is led into the first heat exchanger 34 for cooling down to a temperature lower than −30° C., and in particular of between −40° C. and −70° C.

The second fraction 74 is successively led into the second heat exchanger 36 and reboiler 54 to be cooled therein via heat exchange with the refrigerant circulating in the external refrigerant cycle e.g. propylene and via a reboil stream taken from the bottom of the column 50 respectively.

The second fraction 74 is cooled down to a temperature below −30° C. and in particular of between −40° C. and −70° C.

The ratio of the molar flow rate between the first fraction 72 and the second fraction 74 is controlled by a valve 76, as a function of the available negative calories in the first heat exchanger 34.

The first cooled fraction 72 and the second cooled fraction 74 are then combined to form an initial upstream stream 80.

The initial upstream stream 80 is then led into the third heat exchanger 38 to form an upstream stream 156 cooled to a temperature lower than −40° C., in particular of between −50° C. and −80° C.

The molar content of liquid in the cooled upstream stream 156 is higher than 20% for example and in particular it is between 25% and 50%.

The cooled upstream stream 156 is then led into the upstream separator 40, to be separated therein into an upstream liquid fraction 82 and an upstream gas fraction 84.

The upstream liquid fraction 82 forms a first feed fraction for the column 50 fed into the column 50 at a first level N1 through a flow control valve 86.

The upstream gas fraction 84 is separated into a first upstream gas flow 158 led into the first intermediate heat exchanger 142 and a second upstream gas flow 160 led into the second intermediate heat exchanger 144.

The first upstream gas flow 158 and the second upstream gas flow 160 are each cooled to a temperature lower than −70° C., in particular between −80° C. and −100° C., before being remixed to form a first partly condensed intermediate stream 162.

The molar content of liquid in the first partly condensed intermediate stream 162 is higher than 15% for example and in particular it is between 20% and 35%.

The first partly condensed intermediate stream 162 is then led into the first intermediate separator 148 to be separated therein into a first intermediate liquid fraction 164 and a first intermediate gas fraction 166.

The first intermediate liquid fraction 164 forms a third feed fraction for the column 50 that is fed into the column 50 at a third level N3, located above the first level N1, through a flow control valve 168.

The first intermediate gas fraction 166 is led into the second intermediate heat exchanger 146 to form a second partly condensed intermediate stream 170 cooled to a temperature below −90° C. and in particular of between −100° C. and −130° C.

The second intermediate stream 170 is then led into the second intermediate separator 150 to be separated therein in to a second intermediate liquid fraction 172 and a second intermediate gas fraction 174.

The second intermediate liquid fraction 172 forms a fourth feed fraction for the column 50 fed into the column 50 at a fourth level N4, located above the third level N3, through a flow control valve 176.

The second intermediate gas fraction 174 is led into the first downstream heat exchanger 42 to be cooled therein down to a temperature below −120° C., and in particular of between −125° C. and −150° C. and to form a cooled, partly condensed downstream stream 88.

The molar content of liquid in the downstream stream 88 is higher than 1% for example, and in particular it is between 1% and 20%. The downstream stream 88 is then led into the downstream separator 44 to be separated therein into a downstream liquid fraction 90 and a downstream gas fraction 92.

The downstream liquid fraction 90 forms a second feed fraction for the column 50 and fed into the column 50 at a second level N2, located above the fourth level N4, through a flow control valve 94.

The pressure of the column 50 is lower than 40 kgf/cm², and in particular it is between 5 kgf/cm² and 30 kgf/cm², e.g. lower than 15 kgf/cm².

An ethylene-rich foot stream 96 is recovered at the foot of the column 50. The foot stream 96 has a molar content of ethylene higher than 99.5% and a molar content of carbon monoxide lower than 0.0001 mole % (1 molar ppm). The loot stream 96 contains more than 99 mole % of the ethylene contained in the feed stream 12.

Therefore, the foot stream 96 can be used directly in a polymer production unit without having to be re-distilled.

A head stream 98 depleted of ethylene is extracted at the head of the column 50.

The molar content of carbon monoxide in the head stream 98 is higher than 70%. The head stream 98 contains more than 10 mole % of the carbon monoxide contained in the feed stream 12.

The head stream 98 passes through a flow control valve 110 to form the carbon monoxide-rich downstream flow 106.

The downstream flow 106 is then successively heated in the downstream heat exchanger 42, in each intermediate heat exchanger 146, 142 and then in the upstream heat exchanger 34, via heat exchange respectively with the second intermediate gas fraction 174, the first intermediate gas fraction 166, the first upstream gas fraction 158, and the first fraction 72 of the intermediate stream 20.

The heated downstream flow 180 therefore has a temperature higher than 10° C. on leaving the first upstream heat exchanger 34.

Simultaneously the downstream gas fraction 92 successively passes in the downstream heat exchanger 42, in each intermediate heat exchanger 146, 142, before being led into the dynamic expansion turbine 152 for expansion to a pressure lower than 10 kgf/cm².

The expanded downstream gas fraction 182 formed at the output of the turbine 152 has a temperature lower than −120° C. and in particular between −130° C. and −150° C.

The expanded downstream gas fraction 182 successively enters the downstream heat exchanger 42, each intermediate heat exchanger 146, 142, and then the upstream heat exchanger 34 to be heated therein via heat exchange respectively with the second intermediate gas fraction 174, the first intermediate gas fraction 166, the first upstream gas fraction 158 and the first fraction 72 of the intermediate stream 20.

The heated downstream gas fraction 184 output from the first upstream heat exchanger 34 is led into the first compressor 154 to be compressed therein to a pressure higher than 3 kgf/cm², before optionally being mixed with the heated downstream flow 180.

As for the first installation 10, the second installation 140 of the invention in simple and particularly economical manner allows an ethylene stream 96 to be obtained meeting specifications for polymer production, from a feed stream 12 derived from a non-conventional source and having a high carbon monoxide content.

This result is obtained using a single distillation step performed in a single column 50 in a distillation assembly 18 which also forms a carbon monoxide removing assembly 22.

The recovery of ethylene from the feed stream 112 is practically total, higher than 99%, and the ethylene obtained advantageously has purity higher than 99.5%.

In a first variant of the second installation according to the invention, schematically illustrated FIG. 6, the distillation assembly 18 comprises a head condenser 52 similar to the head condenser 52 of the first installation 10.

The head condenser 52 comprises a head heat exchanger 56, a head separator 58 and a reflux pump 59.

In the example illustrated FIG. 6, the head heat exchanger 56 places the head stream 98 derived from the distillation column 50 in heat exchange contact with the expanded downstream gas fraction 182 output from the dynamic expansion turbine 152, upstream of entry into the downstream heat exchanger 42.

In another variant, a stream of liquid refrigerant circulating in a closed or semi-open refrigeration cycle ensures the production of negative calories in the head heat exchanger 56, via vaporization of refrigerant.

In a second variant of the second installation 140 according to the invention (not illustrated), at least one foot liquid fraction 82, 164, 172 is expanded in a static pressure-reducing valve (not illustrated) to form an expanded foot liquid fraction. The expanded foot liquid fraction is then successively led into the downstream heat exchanger 42, each intermediate heat exchanger 146, 142 and then into the upstream heat exchanger 34, to be heated via heat exchange respectively with the second intermediate gas fraction 174, the first intermediate gas fraction 166, the first upstream gas fraction 158 and the first fraction 72 of the intermediate stream 20.

The heated foot liquid fraction is then recycled in the feed gas 12 upstream of the treatment assembly 14.

In another variant (not illustrated) the mechanical energy collected by the dynamic expansion turbine 152 when expanding the downstream gas flow 92 is dissipated by means of a brake arranged in an oil bath.

In another variant (not illustrated) of the method in FIGS. 1 to 4, the downstream flow 112 is recompressed before being mixed with the feed stream 12.

1. A method to recover an ethylene stream from a feed stream, comprising the following steps:
treating a feed stream to obtain a treated gas stream;
at least partly cooling and condensing the treated gas stream in at least one heat exchanger to form at least one column feed fraction;
feeding the at least one column feed fraction into a distillation column to recover an ethylene stream at the foot of the distillation column and, at the head, a head stream depleted of ethylene;
heating at least one downstream flow derived from the head stream in the heat exchanger;
forming an intermediate stream containing at least 20 mole % ethylene and at least 20 mole % carbon monoxide; and removing the carbon monoxide contained in the intermediate stream.

2. The method according to claim 1, wherein the pressure of the treated gas stream circulating in the at least one heat exchanger is lower than 40 kgf/cm².

3. The method according to claim 2, wherein the pressure of the treated gas stream circulating in the at least one heat exchanger is strictly lower than 30 kgf/cm².

4. The method according to claim 1, wherein the ethylene stream has an ethylene weight content higher than 99.5%, the method comprising obtaining the ethylene stream at the foot of the distillation column without passing through another distillation column.

5. The method according to claim 1, wherein the ethylene stream contains more than 99 weight % of the ethylene contained in the feed stream, the method comprising obtaining the ethylene stream at the foot of the distillation column without passing through another distillation column.

6. The method according to claim 1, comprising leading the intermediate stream into a device to remove carbon monoxide without distillation to form a carbon monoxide-rich stream and a carbon monoxide-depleted stream.

7. The method according to claim 6, wherein the carbon monoxide removing device comprises a pressure swing adsorber producing the carbon monoxide-rich stream and the carbon monoxide-depleted stream.

8. The method according to claim 6, comprising compressing the carbon monoxide-depleted stream to a recompression pressure in a compression equipment to form the treated gas stream.

9. The method according to claim 6, comprising placing at least one fraction of the treated gas stream under heat exchange with the downstream flow in an upstream heat exchanger, to obtain a partly condensed upstream stream, and
leading the upstream stream into an upstream separator and
leading at least one liquid fraction output from the upstream separator into the distillation column.

10. The method according to claim 1, comprising directly forming the intermediate stream by the treated gas stream and then leading said intermediate stream into a condensation and fractionating assembly comprising a plurality of heat exchangers operating at decreasing temperatures to produce a plurality of feed fractions for the distillation column fed into the distillation column the head stream output from the distillation column comprising more than 10 weight % of the carbon monoxide contained in the intermediate stream.

11. The method according to claim 10, comprising:
leading the treated gas stream into at least one first upstream heat exchanger to obtain a cooled and partly condensed upstream stream, and separating the upstream stream into an upstream column feed liquid fraction and an upstream gas fraction;
leading the upstream gas fraction into at least one intermediate heat exchanger to obtain at least one cooled and partly condensed intermediate stream, and separating the at least one cooled intermediate stream into an intermediate column feed liquid fraction and an intermediate gas fraction;
leading at least one intermediate gas fraction into a downstream heat exchanger to obtain a cooled and partly condensed downstream stream, and separating the downstream stream into a downstream column feed liquid fraction and a downstream gas fraction, the upstream, downstream and intermediate liquid fractions being fed into the distillation column.

12. The method according to claim 11, comprising passing at least part of the downstream gas fraction through the downstream heat exchanger and/or through the intermediate heat exchanger and/or through the upstream heat exchanger to be heated therein, the method comprising expanding the heated downstream gas fraction in a dynamic expansion turbine or static pressure-reducing valve before sending back the downstream gas to the downstream heat exchanger and/or intermediate heat exchanger and/or upstream heat exchanger.

13. The method according to claim 11, comprising circulating the downstream flow derived from the head stream in the upstream heat exchanger and/or in the intermediate heat exchanger and/or in the downstream heat exchanger.

14. The method according to claim 1, comprising feeding back, after compression into the feed stream upstream of the treatment, at least one recirculation flow derived from the downstream flow after heating the downstream flow in the heat exchanger.

15. The method according to claim 1, comprising feeding back, after compression into the feed stream upstream of the treatment, at least one recirculation flow derived from the downstream flow after heating the downstream flow in the heat exchanger into the treated gas stream, downstream of the treatment step to form the intermediate stream.

16. An installation to recover an ethylene stream from a feed stream the installation comprising:
an assembly to treat a feed stream to obtain a treated gas stream;
an assembly to cool and at least partly condense the treated gas stream comprising at least one heat exchanger to form at least one column feed fraction;
a distillation assembly comprising a distillation column and an arrangement to feed the at least one column feed fraction into the distillation column to recover an ethylene stream at the foot of the distillation column and an ethylene-depleted head stream at the head of the distillation column;
an assembly to heat at least one downstream flow derived from the head stream in the heat exchanger;
an arrangement to form an intermediate stream containing at least 20 mole % ethylene and at least 20 mole % carbon monoxide;
an assembly to remove the carbon monoxide contained in the intermediate stream; the removing assembly being configured to remove at least part of the carbon monoxide contained in the intermediate stream.

17. The installation according to claim 16, wherein the removing assembly comprises a device to remove carbon monoxide without distillation, to form a carbon monoxide-rich stream and a carbon monoxide-depleted stream.

18. The installation according to claim 16, wherein the removing assembly is formed by the distillation assembly.

19. The method according to claim 6, wherein the pressure of the carbon monoxide depleted stream is lower than 5 kgf/cm².

20. The method according to claim 11, wherein the cooled and partly condensed upstream stream is at a temperature lower than −50° C., the at least one cooled and partly condensed intermediate stream being at a temperature lower than −90° C., the cooled and partly condensed downstream stream being at a temperature lower than −110° C.
